(19) United States
(12) Patent Application Publication
(10) Pub. No.: US 2005/0137552 A1
(43) Pub. Date: Jun. 23, 2005

(54) Shape-adapted absorbent article having improved absorption capacity

(75) Inventors: Morgan Hansson, Vastra Frolunda (SE); Henrik Carlen, Goteborg (SE); Fredrik Asp, Onsala (SE)

Correspondence Address: BURNS DOANE SWECKER & MATHIS L L P, POST OFFICE BOX 1404, ALEXANDRIA, VA, 22313-1404, US

(73) Assignee: SCA Hygiene Products AB, Goteborg (SE)

(21) Appl. No.: 11/019,478

(22) Filed: Dec. 23, 2004

Related U.S. Application Data
(60) Provisional application No. 60/531,611, filed on Dec. 23, 2003.

(51) Int. Cl. A61F 13/15; A61F 13/20

(52) U.S. Cl. 604/385.01

(57) ABSTRACT

An absorbent article with a longitudinal direction and a transverse direction and including an absorption body which is enclosed in a cover and has at least one absorption layer, the cover having a liquid-permeable surface and a liquid-impermeable surface. The absorption body has a second absorption part which is separate from and displaced in the longitudinal direction from a first absorption part. The first absorption part has a tongue-shaped portion adapted to be arranged in between two legs extending essentially in the longitudinal direction on the second absorption part so that fold indications are formed in the absorption body between the tongue-shaped portion and at least each leg. Also relates to a method for manufacturing an absorbent article as described above.

### CROSS-REFERENCE TO RELATED APPLICATIONS

The present application claims the benefit of U.S. Provisional Application No. 60/531,611 filed in the United States on Dec. 23, 2003, the entire contents of which are hereby expressly incorporated herein by reference.

### TECHNICAL FIELD

The invention relates to an absorbent article with a longitudinal direction and a transverse direction and comprising an absorption body which is enclosed in a cover and comprises at least one absorption layer, the cover having a liquid-permeable surface and a liquid-impermeable surface. The invention also relates to a method for manufacturing an absorbent article as described above.

### RELATED ART

Absorbent articles such as diapers, sanitary towels and incontinence shields are intended to catch and absorb body discharges of various kinds. Depending on the area of use and the quantity and nature of body discharge the article is to be capable of absorbing, there are articles of different shape and size. Diapers for heavily incontinent adults, for example, are considerably larger and have greater absorption capacity than diapers for infants. Furthermore, in addition to requirements for sufficient absorption capacity and leakage security, there are also requirements that the articles to be comfortable to wear. For adult wearers, it is also important that the articles are discreet and can be worn without being noticed under normal clothes.

The absorbent articles therefore have to be designed with sufficient absorption capacity to be capable of absorbing body discharge but still have a good fit so that they are pleasant and flexible to wear and so that they follow the body of the wearer closely and prevent leakage. One difficulty in designing an absorbent article intended to be worn in the crotch area of a wearer is that the space between the legs of the wearer is limited. This means that, in order to be comfortable to wear, the article must be narrowest within the area where the majority of the discharge from the wearer will meet the article. There is therefore an obvious risk that that area of the article which is affected first by body discharge will be oversaturated and will not be capable of absorbing any more when the next evacuation takes place. This means that the risk of leakage is already great when the article has absorbed relatively small quantities of body discharge in relation to its total capacity. In order to prevent leakage past the side edges of an absorbent article of this kind, it is therefore common to provide the article with some type of edge barrier. Such edge barriers are in most cases elastic and form raised physical obstacles to the passage of discharges. On diapers and incontinence shields of the kind which is worn like a pair of absorbent underpants, it is common to arrange elastic elements which fit tightly around the legs of the wearer and in this way hold the edges of the article in sealing contact with the legs.

On articles such as diapers and incontinence shields for heavily incontinent people, elastic leg bands and raised barriers are usually combined with the article being designed with a relatively wide crotch portion so as to bring about sufficient absorption capacity within the wetting area of the article. During use, such a crotch portion will be folded together between the legs of the wearer or will hang down as a liquid-collecting bag between the legs. Such a design creates random channels, which can give rise to leakage, is not especially comfortable or discreet and moreover functions poorly when the wearer sits down. For sanitary towels and other absorbent articles where the requirement for discretion during use is particularly high, such an unwieldy and inconvenient construction is not at all acceptable. Articles such as sanitary towels and incontinence shields for mildly incontinent people are moreover rather small and not self-supporting but are fastened inside a pair of ordinary briefs which hold the article in contact with the body of the wearer during use. A construction with a crotch portion which hangs down or is folded together cannot therefore be used for this category of article.

In order to manufacture shape-adapted and size-adapted absorbent articles with varying absorption capacity, a method is also required in which most of the individual manufacturing steps have to be adapted in order to provide articles of suitable shape and size. In this connection, it is also usually necessary to exchange most of the components required for the manufacturing method in order for it to be possible to obtain the shape-adapted and size-adapted articles. In manufacturing, resetting equipment represents an operation which on the one hand results in a technically more complicated process and on the other hand adds extra costs in the form of machine components and costs for resetting times. As cost is a critical factor in the manufacture of disposable absorbent articles, such as shape-adapted absorbent articles, it is essential that absorbent articles of this kind can be manufactured in a simple and effective way so that the production cost and, at the next stage, the selling cost can be kept low.

As is evident from the above, there is a need for an absorbent article which has a good fit, high leakage security, high absorption capacity in the area which is affected first by body discharge, and which is discreet and comfortable to wear. It is also desirable to be able to provide an absorbent article which has such a good fit that special shaping elements or leakage barriers, for example in the form of elastic elements, can be avoided. There is moreover a need for a simple and cost-effective method for manufacturing the absorbent article.

### OBJECTS AND SUMMARY

By means of embodiments of the present invention, an article has been produced with a longitudinal direction and a transverse direction and comprising an absorption body enclosed in a cover, the cover having a liquid-permeable surface and a liquid-impermeable surface, and the absorption body comprising at least one absorption layer, which article has a good fit, high leakage security, high absorption capacity, and high discretion and comfort.

An article made according to one embodiment of the invention is characterized mainly in that the absorption body comprises a first absorption part which is separate from a second absorption part. The first absorption part has a tongue-shaped portion adapted to be arranged in between two legs extending essentially in the longitudinal direction on the second absorption part so that fold indications are formed in the absorption body between the tongue-shaped portion of the first absorption part and at least each leg of the second absorption part.

By folding the first and second absorption parts, respectively, in relation to one another along the fold indications formed between the first and second absorption parts, a curvature of the absorption body is brought about in at least the area where the tongue-shaped portion of the first absorption part lies adjacent to each leg of the second absorption part. The curvature is therefore formed by virtue of the fact that, when folding takes place in the fold indications, each leg of the second absorption part is folded in relation to the tongue-shaped portion of the first absorption part so that a bowl-shape is formed with the legs constituting sides of the bowl. In this way, the curved absorption layer is highly suitable as a component in an absorbent article, for example a diaper, a sanitary towel, an incontinence shield or the like, as the curved shape essentially follows the curvature of the body of the wearer. Absorbent articles of this kind usually have two end portions and a crotch portion which is located between the end portions and often has a smaller size in the transverse direction than the end portions. In this connection, it is especially advantageous that the fold indications in the absorption body are arranged at least mainly in the crotch portion. The fold indications are suitably formed in the absorption body between the tongue-shaped portion of the first absorption part and at least each leg of the second absorption part on both sides of a centre line extending along the article in the longitudinal direction. In this connection, the fold indications extending in the longitudinal direction are suitably arranged at a distance from one another in the transverse direction which essentially corresponds to the width of the crotch area of a wearer.

In order to obtain an absorbent article, as an embodiment of the invention, it is suitable that the two legs extending in the longitudinal direction on the second absorption part have a length, s, of 3-30 cm, preferably 5-25 cm, and most preferably 7-20 cm and/or that the tongue-shaped portion of the first absorption part has a length, t, of 3-30 cm, preferably 5-25 cm, and most preferably 7-20 cm.

For diapers for children, it is suitable that each fold indication extending in the longitudinal direction has a length of 3-20 centimeters, preferably about 15 centimeters, and that the spacing in the transverse direction between the fold indications extending in the longitudinal direction has a width of 1-5 centimeters, preferably about 3 centimeters. For diapers and other types of body-enclosing incontinence shields for adults, it is suitable that each fold indication extending in the longitudinal direction has a length of 3-30 centimeters, preferably about 20 centimeters, and that the spacing in the transverse direction between the fold indications extending in the longitudinal direction has a width of 1-7 centimeters, preferably about 4 centimeters. In this connection, the length, l, extending in the longitudinal direction, of a fold indication is the distance between the point which essentially corresponds to the outermost tip of a leg and the termination point of the leg. In this connection, the termination point of a leg is essentially located where the inner edge of a leg starts to merge with the U-shaped inner edge of the second absorbent part, which U-shaped inner edge lies between the two termination points. The width of the folded portions is the distance in the transverse direction, measured at the termination points, between the fold edges formed at the respective fold indications. For sanitary towels, or incontinence shields for mildly incontinent people, the fold indications suitably have a length between the end points of 5-20 centimeters, preferably about 10 centimeters, and the folded portions have a width of 1-5 centimeters, preferably 2.5 centimeters.

In order to obtain a more accentuated fold indication, there can be a distance of 1-20 mm, preferably 2-15 mm, and most preferably 3-10 mm, between the tongue-shaped portion of the first absorption part and each leg of the second absorption part, at least at the outer parts of the legs. Here, the outer parts of the legs is the whole of the length, l, extending in the longitudinal direction, of the fold indication, preferably half the length, l, displaced at the outermost tip of the leg concerned, and most preferably a third of the length, l, displaced at the outermost tip of the leg concerned. In addition to the advantage that it will thus be possible for the fold indication to allow a more marked fold between the parts located on each side of the fold indication, a space which is free from absorbent material will also be provided between the first absorption part and each leg of the second absorption part. This free space is advantageous in particular when a large quantity of liquid is discharged in a short time. Liquid can then be caught rapidly in this free space, to be subsequently absorbed by surrounding material. The advantages of this embodiment become even more apparent after a first wetting, that is to say on subsequent wettings when further discharged liquid has to be distributed in the material of the core parts, which is already wet and may have swollen owing to liquid absorption.

However, in another embodiment the tongue-shaped portion of the first absorption part and each leg of the second absorption part can also, at least at the outer parts of the legs, be arranged so that the first absorption part and the second absorption part lie edge to edge with one another, so that a more shape-adapted product is then obtained.

As mentioned above, the first absorption part is designed with a tongue-shaped portion, so that it is possible for the tongue-shaped portion to have an essentially U-shaped outer edge. The first absorption part also extends from the tongue-shaped portion in the longitudinal direction and/or in the transverse direction so that a large free surface is formed. The large free surface is preferably larger than the surface of the tongue-shaped portion.

The second absorption part is designed so that its legs have an inner edge which comes in towards a central line in the longitudinal direction in the second absorption part so as thus to form an essentially U-shaped edge. The second absorption part also extends from its legs in the longitudinal direction and/or in the transverse direction so that a large free surface is formed. In this connection, the second absorption part has a tooth-like appearance where the legs may be said to correspond to the two roots of the tooth. In one embodiment, the legs preferably comprise the same absorption material as the free surface of the second absorption part. In order to increase the dimensional stability, however, the legs advantageously also comprise stiffening material in one embodiment.

When the first absorption part, according to one embodiment, is adapted to the second absorption part so that fold indications are formed in the absorption body between the tongue-shaped portion of the first absorption part and at least each leg of the second absorption part, the fold indications will be most distinctly marked where the first absorption part and the second absorption part are arranged with a free space between them, or at least lie edge to edge.

However, a less marked fold indication will also be formed in the situation where the first absorption part lies overlapping the second absorption part. The outer edge on the tongue-shaped portion of the first absorption part will then, during use, be pressed against the second absorption part and form a fold indication along which the second absorption part will be folded. The specifically mutually adapted shapes of the first absorption part and the second absorption part will consequently produce a curved shape which extends in both the transverse direction and the longitudinal direction over the crotch area. The embodiment of the invention as described above therefore provides a three-dimensional curved shape which can be adapted optimally to the crotch area of a wearer and which therefore brings about both a good fit and increased leakage security.

Depending on how accentuated the required folding is, the first absorption part and the second absorption part can be arranged in relation to one another so that a more marked fold is achieved. According to a preferred embodiment of the invention, there is a distance between the U-shaped edge of the second absorption part and the tongue-shaped portion of the first absorption part of 0-20 mm, preferably 2-15 mm, and most preferably 3-10 mm, along the whole tongue-shaped portion. According to another embodiment, an inner edge of the legs of the second absorption part comes in towards a longitudinal centre line so as thus essentially to correspond to and essentially to run edge to edge with the outer edge of the tongue-shaped portion of the first absorption part.

As mentioned above, the tongue-shaped portion of the first absorption part can overlap at least a part of the second absorption part. A less marked bowl shape of the absorption body will thus be obtained in this embodiment. However, there is also an advantage in an area with an overlap being formed. Such an overlap is then especially advantageous when increased absorption capacity is desired in parts of the absorbent article which will be subjected to great loading by body discharges. According to a preferred embodiment of the invention, the overlap is such that the tongue-shaped portion of the first absorption part overlaps a part of the second absorption part so that a double layer is formed in at least an area corresponding to the wetting zone of the absorbent article. The wetting zone is the zone which lies adjacent to the genitals of the wearer when the absorbent article is used.

According to one embodiment of the invention, the first absorption part is a rear absorption part located in a rear end portion of the article, which is adapted to face backwards on the wearer during use, and the second absorption part is a front absorption part located in a front end portion of the article, which is adapted to face forwards on the wearer during use. However, the first absorption part can be a front absorption part located in a front end portion of the article, and the second absorption part can be a rear absorption part located in a rear end portion of the article.

The front and rear absorption parts can preferably comprise absorbent material of the same kind. The front absorption part can preferably also be designed so that it comprises absorbent material specially adapted for taking up urine and/or other viscous liquids such as menstrual fluids and discharges. The rear absorption part can also be designed so that it comprises absorbent material adapted for taking up feces and/or other viscous liquids such as menstrual fluids and discharges. A combination of a front absorption part adapted mainly for urine absorption and a rear absorption part adapted for taking up mainly feces is especially preferred when the front absorption part and the rear absorption part are separated from one another. Here, separated means that the parts constitute two parts which are independent of one another, but can still lie overlapping one another, edge to edge, or spaced apart. When the front and rear absorption parts comprise material adapted specially for their respective take-up and when they are, in addition, preferably not interconnected by any liquid-conducting material, the different body discharges can be kept separated and odor can be avoided. A mixture of urine and fecal matter is moreover particularly aggressive as far as action on the skin is concerned.

The front and rear absorption parts are advantageously movable in relation to one another to such an size that a curved shape as described above can be formed. However, the first absorption part can be locked in a predetermined position in relation to the second absorption part by a stabilizing means, without the mobility between the two parts being lost. In a case where the stabilizing means is a stabilizing layer which is connected firmly to the first absorption part and the second absorption part, folds will still be formed in the fold indications. The stabilizing layer can be attached along the whole surface of the first absorption part and the second absorption part, respectively, or along parts thereof.

The stabilizing layer can constitute a distance layer between the first absorption part and the second absorption part, respectively, and comprise either the liquid-permeable surface of the cover or the liquid-impermeable surface of the cover. Alternatively, the stabilizing layer can comprise the liquid-permeable surface of the cover or of the liquid-impermeable surface of the cover, or both cover layers. When either or both of the cover layers are designed to stabilize the absorption body, each absorption part can be attached completely or partly to the layer(s) concerned depending on how great the mobility required is. The stabilizing layer can be attached by, for example, gluing or welding. The stabilizing layer can comprise, for example, in addition to a liquid-permeable cover layer or a liquid-impermeable cover layer, a layer which in a finished absorbent article functions as a strengthening layer, a liquid-receiving layer or an additional absorption layer.

As mentioned above, a method for manufacturing shape-adapted absorbent articles with improved absorption capacity where the individual manufacturing steps can be adapted in order simply and effectively to provide articles of suitable shape and size is also particularly desirable.

A method for producing an absorption body made according to embodiments of the invention for use in an absorbent article is characterized mainly in that the absorption body is produced by a first absorption part being shaped with a tongue-shaped portion and a second absorption part being shaped with two legs extending in the longitudinal direction. The first absorption part is positioned separately and displaced in the longitudinal direction in connection to the second absorption part so that the tongue-shaped portion of the first absorption part is arranged completely or partly in between the two legs extending essentially in the longitudinal direction on the second absorption part, fold indications then being formed in the absorption body between the tongue-shaped portion of the first absorption part and at least each leg of the second absorption part.

In manufacture, at least a part of the inner edge of the legs of the second absorption part can preferably be positioned edge to edge with the outer edge of the tongue-shaped portion. The first absorption part can alternatively be applied to the second absorption part so that the tongue-shaped portion of the first absorption part overlaps at least a part of the second absorption part.

Normally, more than one size of absorbent article is manufactured on one and the same production machine. When the size of the article is to be changed, a number of machine components such as, for example, profile-cutting units, compression units or the like have to be exchanged in this connection. For an absorbent article according to embodiments of the invention, however, a smaller number of machine components have to be exchanged. Above all, the mat-forming moulds of the machine, where the absorption bodies are produced, do not have to be exchanged, especially if the change in size is relatively small. When the size of the article is to be changed in a manufacturing machine which produces absorbent articles according to embodiments of the invention, the first and second absorption parts are instead arranged overlapping one another to a greater or lesser extent or completely separated in the crotch portion of the absorbent article, the size then being changed. The time-consuming exchange of mat-forming moulds is then eliminated, making it possible for both the resetting time and the number of persons involved in the resetting process to be reduced significantly.

When the size of the article is changed, the intermixing of gel-forming material, what are known as superabsorbents, or the quantity of pulp fibers in the moulds can be increased/decreased by the material being pressed together to a greater or lesser extent in the moulds, it then being possible to adjust the absorption capacity of the article. The same advantage is also obtained in the manufacture of absorption bodies which are cut from a web-shaped absorption material. The absorption capacity can then be changed by selecting a web-shaped material comprising more or less superabsorbent material, and increasing or decreasing the weight per unit area of the web-shaped material.

The shape of the absorption body after positioning of the first absorption part and the second absorption part in relation to one another can also be fixed by joining the absorption body together with a stabilizing layer, for example by gluing or welding. In this connection, the shape of the absorption body can be fixed by activating a bonding means included in the absorption body, or by gluing or welding, to a layer included in the absorbent article, such as, for example, a stabilizing layer and/or one or both of the cover layers. According to one embodiment of the invention, the bonding means comprises thermoplastic fibers, and the shape of the absorption body is stabilized by heating and subsequent cooling of the absorption body.

### DESCRIPTION OF FIGURES

Embodiments of the invention will be described in greater detail below with reference to the illustrative embodiments shown in the accompanying drawings, in which:

FIG. 1 shows a diaper according to an embodiment of the invention;

FIG. 2a shows a section along the line II-II through the diaper in FIG. 1 according to a first embodiment of the invention;

FIG. 2b shows a section along the line II-II through a diaper in FIG. 1 in wearer position;

FIG. 3 shows an absorption body according to an embodiment of the invention, and

FIG. 4 shows another absorption body according to an embodiment of the invention.

### DETAILED DESCRIPTION OF THE PREFERRED EMBODIMENTS

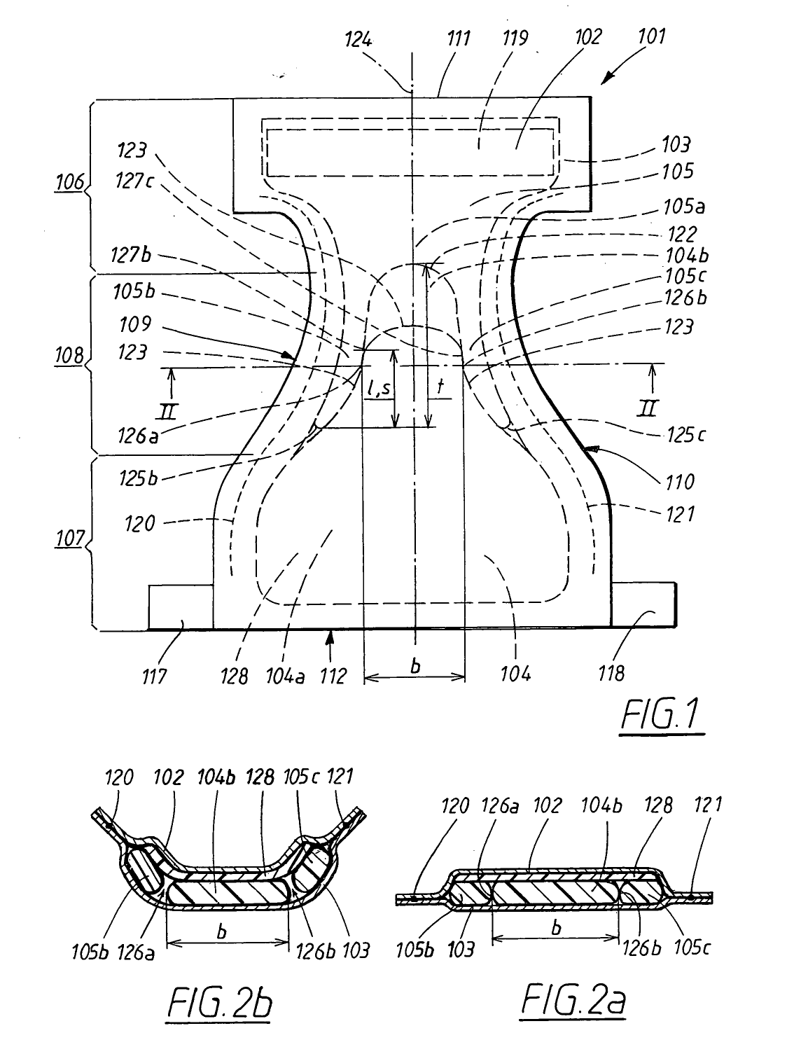

An embodiment of the invention is the diaper 101 shown in FIG. 1, which comprises a first, liquid-permeable cover layer 102, a second, liquid-impermeable cover layer 103, and an absorption body which is arranged between the cover layers 102, 103 and comprises a first absorption part 104 and a second absorption part 105. The two cover layers 102, 103 have a greater size in the plane than the absorption body and project beyond the absorption body around its whole periphery. The cover layers 102, 103 are interconnected at the projecting portions, for example by means of gluing or welding using heat or ultrasound.

The liquid-permeable cover layer 102 can comprise any suitable material such as layers of nonwoven material, perforated plastic film, net material, tow (parallel continuous fiber threads) or the like. The cover layer 102 can also comprise a laminate of two or more layers of the same or different material. Within the scope of the invention, however, the liquid-permeable cover layer 102 does not have to be a separate component but can be an integrated part of the absorption body 104, 105. In such an embodiment, the liquid-permeable cover layer 102 is not necessarily included in the projecting edge around the absorption body 104. Examples of materials which can both be the cover layer 102 and form part of the absorption body 104 are foam, fiber wadding, nonwoven material or the like.

The liquid-impermeable cover layer 103 can comprise a liquid-impermeable plastic film, a nonwoven layer which has been coated with a liquid-blocking material, or another flexible material layer capable of withstanding liquid penetration. It can be an advantage, however, if the liquid-impermeable cover layer 103 has a certain breathability, that is, it allows the passage of water vapor through the layer 103. Like the liquid-permeable cover layer 102, the liquid-impermeable cover layer 103 can be an integrated part of the first absorption part 104 and the second absorption part 105 of the absorption body and can comprise, for example, a liquid-impermeable skin-like surface on an absorbent foam material. If both the liquid-permeable and the liquid-impermeable cover layers 102, 103 constitute integrated parts of the absorption body, the first absorption part 104 and the second absorption part 105 included in the absorption body should be stabilized by means of a stabilizing layer.

The diaper 101 has an elongated shape, with wider front and rear portions 106, 107 and a narrower, intermediate crotch portion 108. In this connection, the front portion 106 is that part of the diaper 101 which is adapted to face forwards on the wearer when the diaper is used, and the rear portion 107 is that part of the diaper which faces backwards on the wearer. The diaper 101 also has two longitudinal, inwardly curved side edges 109, 110, a front edge 111 and a rear edge 112.

The diaper 101 is of the kind which is fastened together when it is used, so that it surrounds the lower part of the abdomen of the wearer in a pant-like manner. For this purpose, a tape flap 117, 118 is arranged projecting from each side edge 109, 110, close to the rear edge 112 of the diaper. The tape flaps 117, 118 are adapted to interact with a receiving area 119 arranged on the liquid-impermeable cover layer 103 on the front portion 106 of the diaper 101. Such a receiving area 119 suitably involves some form of strengthening of the liquid-impermeable cover layer 103, for example in the form of an extra plastic layer or a coating applied to the liquid-impermeable cover layer 103. It is of course alternatively conceivable to use other types of fastening devices for the diaper 101, such as buttons and buttonholes, hooks and eyes, press studs, hook-and-loop fasteners or the like. When hook-and-loop fasteners are used, these can suitably be arranged so that the male parts of the hook-and-loop fasteners are arranged as corresponding flaps 117, 118 and the female parts as a corresponding receiving area 119. Here, the male parts are the hook-shaped elements of the hook-and-loop fastener, and the female parts are the loop-shaped elements of the hook-and-loop fastener. However, the flaps 117, 118 can also comprise female parts and the receiving area 119 male parts. Another alternative is for the diaper to be what is known as a pant diaper, which means that it is supplied to the wearer as continuous absorbent pants adapted to be pulled over the legs. Pant diapers can also be openable. Another common diaper type is fastened together by means of a belt to which the diaper is attached or from which it is detachably suspended.

The diaper 101 is also provided with pretensionally arranged, essentially longitudinal elastic elements 120, 121, arranged along the side edges 109, 110 of the diaper. The elastic elements 120, 121 contribute to curving the diaper 101 according to the body of the wearer during use and at the same time constitute the leg elastic of the diaper. In this way, the elastic elements 120, 121 serve to hold the side edges 109, 110 of the diaper in sealing contact with the legs of the wearer so as to counteract gaps appearing between the diaper and the body of the wearer during use, through which body fluid can leak out of the diaper. The elastic elements are not necessary for this embodiment of the invention and can be omitted because an absorption body according to embodiments of the invention, and the diaper 101, typically have a good fit and follow the body of the wearer closely even without elastic elements 120, 121.

The first absorption part 104 of the absorption body is arranged in an overlap over the second absorption part 105 so that a double layer of absorption material is formed in the area which will correspond to the wetting zone of the absorbent article. The first absorption part 104 is designed with a large free surface 104a and a tongue-shaped portion 104b which has an essentially U-shaped outer edge 122. The tongue-shaped portion 104b has a length, t, shown in the figure. The second absorption part 105 is designed with a large free surface 105a which extends to two legs 105b, c. The absorption body is designed so that the free surface 104a of the first absorption part 104 will during use constitute part of the rear portion 107, and the free surface 105a of the second absorption part 105 will constitute part of the front portion 106. The tongue-shaped portion 104b and the legs 105b, c will constitute part of the crotch portion 108 lying between the front portion and the rear portion. The legs 105b, c have an inner edge 123 which comes in towards the free surface 105a of the second absorption part 105, the inner edge 123 also forming an essentially U-shaped edge. The two legs 105b, c extend essentially in the longitudinal direction along a longitudinal centre line 124 in the diaper 101 towards the outer tip 125b, c of the respective leg. Two fold indications 126a, b formed in essentially the longitudinal direction run at least between the outer tip 125b, c of the respective leg and the respective termination point 127b, c, which points are located essentially where the inner edge 123 of the legs starts to merge with the U-shaped edge of the second absorbent part. The distance between the outer tip 125b, c of each leg and the corresponding respective termination point 127b, c is indicated in FIG. 1 as a length l. In this embodiment, the length, l, corresponds essentially to the length, s, of the legs. Along the length, l, a part of the tongue-shaped portion 104b will lie edge to edge with the respective leg 105b, c, a marked fold indication part 126a, b being formed in this part. As the tongue-shaped portion 104b overlaps a part of the second absorption part 105, however, the fold indication 126a, b will also merge with a less marked portion. This less marked fold indication part 126a, b is formed by the outer edge 122 on the tongue-shaped portion 104b. The fold indications 126a, b will during use form a more marked fold along the length 1, then merging with a less marked fold along the outer edge 122 of the tongue-shaped portion 104b as the latter is pressed against the second absorption part 105. As a whole, the fold indications 126a, b will during use make possible a curved, anatomically adapted shape fitted to the crotch portion of the wearer. The crotch portion of the absorbent article will during use have a width, b, which will essentially correspond to the distance in the transverse direction, measured at the termination points 127b, c, between the fold edges formed at the respective fold indications 126a, b.

By virtue of the overlap and the legs 105b, c which are bent during use, a thicker, more absorbent portion is obtained in that area of the diaper 101 which is expected to be wetted first by body fluid, at the same time as the crotch portion 108 can be made considerably narrower than is possible in conventional diapers. The extra quantity of absorption material is localized to a small, relatively narrow area of the crotch portion 108, which makes the diaper comfortable to wear in spite of the increased absorption capacity. The folded legs 105b, c also result in stabilization and stiffening of the crotch portion 108 and in this way counteract uncontrolled deformation and creasing, contributing to increased comfort and to the diaper being discreet to wear and leakproof.

The first absorption part 104 and the second absorption part 105 of the absorption body can be constructed from one or more layers of absorbent material, such as cellulose fluff pulp, tissue, absorbent foam, etc. It is also common for the absorption body to contain superabsorbents, that is to say polymer materials which can absorb body fluid corresponding to several times their own weight while forming a hydrogel. Such superabsorbents are usually in the form of particles, but fibers, flakes, granulates and film are also found. The two parts 104, 105 of the absorption body can also comprise non-absorbent components such as stiffening elements, shaping elements, bonding means, etc. Various types of liquid-receiving porous structures such as fiber waddings or the like can also be included in the diaper 101. In particular, the legs 105b, c and/or the outer edge 122 on the tongue-shaped portion 104b can advantageously comprise stiffening elements and/or shaping elements in order to increase the dimensional stability in the fold indication 126a, b.

The first and second absorption parts 104, 105 can preferably comprise absorbent material of the same kind. Alternatively, the first and second absorption parts 104, 105 can comprise absorbent materials specially adapted for taking up feces and urine respectively, which is known within the art. The absorbent material can also be specifically adapted so as also to be capable of taking up viscous liquids such as menstrual fluids and discharges.

The diaper 101 also comprises a liquid-transfer layer 128, which is arranged between the liquid-permeable cover layer 102 and the two parts 104, 105 of the absorption body. In the illustrative embodiment shown, the liquid-transfer layer 128 is the same shape and size as the absorption body, which is of course not necessary for the invention. The liquid-transfer layer 128 can therefore be smaller or larger than the absorption body and have a shape different to that of the absorption body.

The liquid-transfer layer 128 between the liquid-permeable cover layer 102 and the absorption body suitably has higher porosity than the absorption body and has a good capacity for receiving liquid and for giving liquid up to the absorption body. The liquid-transfer layer 128 also constitutes a distance layer between liquid which has been absorbed in the absorption body and the body of the wearer and in this way prevents rewetting. That is, the distance layer prevents body fluid already absorbed from escaping back out through the liquid-permeable cover layer 102. In general, it is an advantage if the liquid-transfer layer 128 comprises a thermoplastic component which can be used in order to join the liquid-transfer layer 128 together with the liquid-permeable cover layer 102 and/or the absorption body. In this regard, the liquid-transfer layer 128 advantageously forms a stabilizing layer which fixes the first and second parts 104, 105 of the absorption body in relation to one another.

FIG. 2a shows a section along the line II-II through the diaper in FIG. 1. In FIG. 2a, the liquid-transfer layer 128 extends over the first leg 105b of the first absorption part, the tongue-shaped portion 104b and the second leg 105c of the first absorption part. The shape of the absorption body is locked by joining the tongue-shaped portion 104b together with the liquid-transfer layer 128. The joining can be effected in any suitable way, for example by gluing, needling or welding or fusing thermoplastic components in the liquid-transfer layer 128 together. The liquid-transfer layer 128 positions only the legs 105b, c in a physical respect and is therefore not joined to the legs 105b, c. When the diaper 101 is folded along fold indications 126a, b, the legs 105b, c will therefore be freely movable in relation to the tongue-shaped portion 104b. It is also possible to fix the shape of the diaper by joining the absorption body together with the liquid-impermeable cover layer 103 or, if the liquid-transfer layer 128 is excluded, with the liquid-permeable cover layer 102.

An important advantage of the absorption body shown in FIG. 2a is that, when discharged liquid has been transported past the liquid-transfer layer 128, a certain quantity of liquid can easily pass through liquid-admission channels for rapid transport of liquid into the absorption body. The liquid-admission channels are formed by the fold indications 126a, b. The liquid can also be spread rapidly in the longitudinal direction of the diaper 101 in the fold indications 126a, b, and then be absorbed by parts of the absorbent material which are more peripheral than the wetting point.

FIG. 2b shows a section along the line II-II through a diaper in FIG. 1 when the diaper is curved in accordance with the wearer position. Like the section of the diaper shown in FIG. 2a, a liquid-transfer layer 128 is shown, which extends over the first leg 105b of the first absorption part, the tongue-shaped portion 104b and the second leg 105c of the first absorption part. In a way corresponding to that described in FIG. 2a, the shape of the absorption body is locked by joining the tongue-shaped portion 104b together with the liquid-transfer layer 128. As can be seen from FIG. 2b, the liquid-transfer layer 128 positions only the legs 105b, c in a physical respect and is not joined to the legs 105b, c. When the diaper 101 is folded along fold indications 126a, b, the legs 105b, c are freely movable in relation to the tongue-shaped portion 104b. In this connection, the liquid-admission channels for rapid transport of liquid into the absorption body are formed in the fold indications 126a, b. The width of the liquid-admission channels formed can be adjusted by the liquid-transfer layer 128 being joined together with the legs 105b, c at a suitable distance from the tongue-shaped portion 104b. It is also possible, as described above, to fix the shape of the diaper by joining the absorption body together with the liquid-impermeable cover layer 103 or, if the liquid-transfer layer 128 is excluded, with the liquid-permeable cover layer 102.

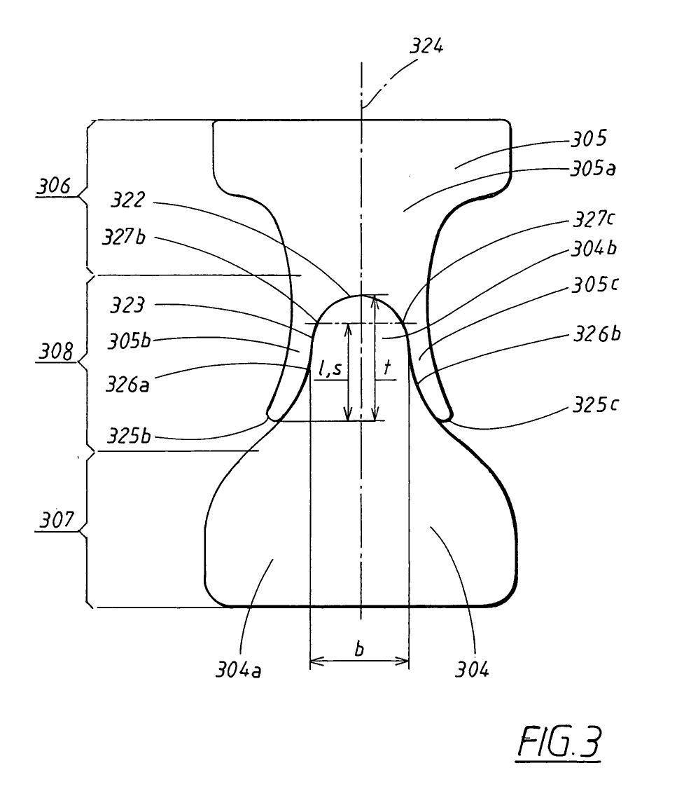

An embodiment of the invention, the absorption body shown in FIG. 3, has the same basic construction as the absorption body in FIGS. 1 and 2 and therefore comprises a first absorption part 304 and a second absorption part 305. The first absorption part 304 has a large free surface 304a and a tongue-shaped portion 304b which has an essentially U-shaped outer edge 322. The tongue-shaped portion 304b has a length, t, shown in the figure. The second absorption part 305 has a large free surface 305a which runs out into two legs 305b, c. The absorption body is designed so that the free surface 304a of the first absorption part 304 will during use constitute part of the rear portion 307, and the free surface 305a of the second absorption part 305 will constitute part of the front portion 306. The tongue-shaped portion 304b and the legs 305b, c will constitute part of the crotch portion 308 lying between the front portion and the rear portion. In this regard, the front portion 306 is that part of the absorption body which is adapted to face forwards on the wearer when the absorption body is used, and the rear portion 307 is that part of the absorption body which faces backwards on the wearer. The legs 305b, c have an inner edge 323 which comes in towards the free surface 305a, the inner edge 323 having essentially a U-shape. The two legs 305b, c extend essentially in the longitudinal direction along a longitudinal centre line 324 towards the outer tip 325b, c of the respective leg. Two fold indications 326a, b formed in essentially the longitudinal direction run between the outer tip 325b, c of the respective leg and the respective termination point 327b, c, which points are located essentially where the inner edge 323 of the legs starts to merge with the curvature of the U-shaped part. The distance in the longitudinal direction between the outer tip 325b, c of each leg and the corresponding respective termination point 327b, c is indicated in the figure as a length l. This fold indication length, l, has a slightly longer size in the longitudinal direction than the corresponding fold indication length, l, shown in FIG. 1, which gives rise to more accentuated folding of the absorbent body taking place over the crotch portion. In this embodiment as well, the length, l, corresponds essentially to the length, s, of the legs. In this embodiment, the whole outer edge 322 of the tongue-shaped portion 304b lies edge to edge with the whole inner edge 323, which means that the fold indication will run along the whole of the U-shaped part of the tongue-shaped portion. More accentuated folding will then be obtained in both the transverse direction and the longitudinal direction of the absorption body, which leads to a more distinct bowl shape. A deeper bowl shape has been found to be advantageous inter alia as it retains liquid better even when the wearer is lying on a side.

The embodiment shown in FIG. 3 is also especially advantageous when the absorption body is adapted for separate take-up of urine and feces. By virtue of the first and second absorption parts being more separated from one another than in the embodiment shown in FIG. 1, it is more difficult for body discharges to be transferred between the absorption parts. The odor which arises when different body discharges meet can thus be avoided.

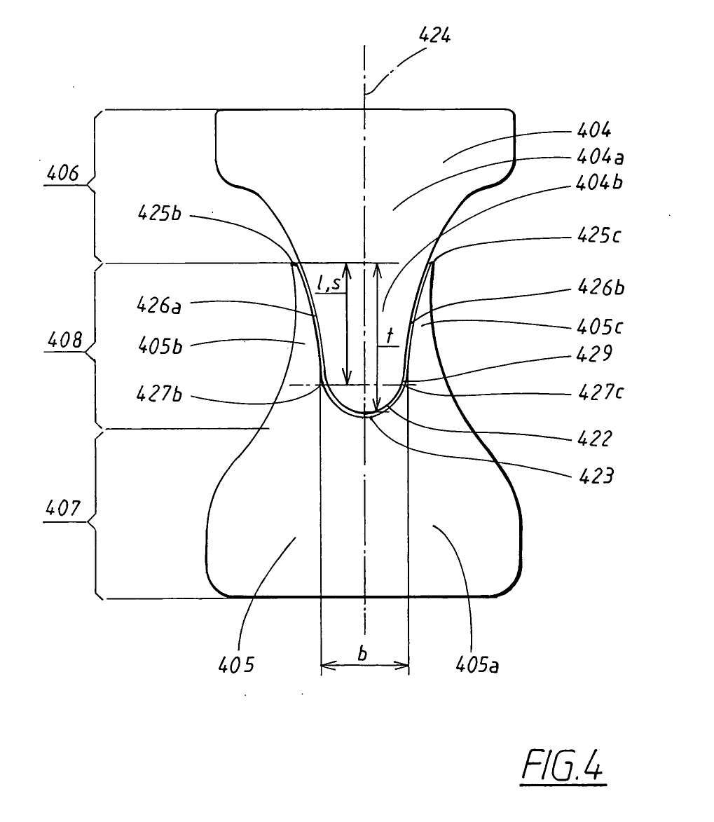

FIG. 4 shows an embodiment of the invention, an absorption body, which, like the absorption bodies shown in FIGS. 1 and 3, has a first absorption part 404 and a second absorption part 405. The first absorption part 404 also has a large free surface 404a and a tongue-shaped portion 404b which has an essentially U-shaped outer edge 422. The tongue-shaped portion 404b has a length, t, shown in the figure. The second absorption part 405 has a large free surface 405a which runs out into two legs 405b, c. In contrast to the absorption bodies shown in FIGS. 1 and 3, the absorption body is in this embodiment positioned so that the free surface 404a of the first absorption part 404 will during use constitute part of the front portion 406, and the free surface 405a of the second absorption part 405 will constitute part of the rear portion 407. The tongue-shaped portion 404b and the legs 405b, c will constitute part of the crotch portion 408 lying between the front portion and the rear portion. In this connection, the front portion 406 is that part of the absorption body which is adapted to face forwards on the wearer when the absorption body is used, and the rear portion 407 is that part of the absorption body which faces backwards on the wearer. The legs 405b, c also have an inner edge 423 which comes in towards the free surface 405a, the inner edge 423 having essentially a U-shape. The two legs 405b, c extend essentially in the longitudinal direction along a longitudinal centre line 424 towards the outer tip 425b, c of the respective leg. Two fold indications 426a, b formed in essentially the longitudinal direction run between the outer tip 425b, c of the respective leg and the respective termination point 427b, c along a length, l, and also along the whole inner edge 423 along the U-shaped part. In this embodiment, the length, l, corresponds essentially to the length, s, of the legs. In this embodiment, the whole outer edge 422 of the tongue-shaped portion 404b is fixed at a distance of about 5 mm from the whole inner edge 423, which means that the fold indication will run along the whole of the U-shaped part of the tongue-shaped portion 404b. Even more accentuated folding will then be obtained in both the transverse direction and the longitudinal direction of the absorption body, which leads to a more distinct bowl shape.

One advantage of this embodiment is that a space 429 which is free from absorbent material is provided between the tongue-shaped portion 404b and each leg 405b, c. This free space 429 is advantageous as it functions as a channel in particular when a large quantity of liquid is discharged in a short time. As described above, liquid can then be caught rapidly in this free space, subsequently being absorbed by surrounding material. The advantages of this embodiment also become even more apparent after a first wetting, that is to say on subsequent wettings when further discharged liquid has to be distributed in the material of the core parts, which is already wet and may have swollen owing to liquid absorption. In the illustrative embodiment shown, the free space 429 is free from material. However, it is conceivable for the free space 429 to be completely or partly filled with a porous material, for example wadding or foam.

The embodiment shown in FIG. 4 is preferred when the absorption body is adapted for separate take-up of urine and feces. By virtue of the first and second absorption parts being spaced apart from one another compared with the embodiments in FIGS. 1 and 3, it is not possible for body discharges to be transferred between the absorption parts. The odor which arises when different body discharges meet can thus be avoided altogether. Moreover, as the mixture of urine and fecal matter is particularly aggressive as far as action on the skin is concerned, such negative effects can also be avoided. In this connection, the positioning of the first absorption part 404 in the front portion 406 is especially advantageous as the tongue-shaped portion 404b can here be given such dimensions that the wetting zone is comprised in the first absorption part 404.

When an absorption body according to FIG. 4 is used in a diaper, the diaper may come to lie in close contact with the body of the wearer owing to pressure from the wearer. In this connection, a distance layer is suitably arranged between the liquid-permeable cover layer and the absorption body 404. The risk of liquid taken up in the free space 429 being pressed back so that what is known as rewetting occurs is then reduced.

Although embodiments of the invention have been described above in connection with diapers, it is of course possible to apply the invention to absorbent articles such as incontinence shields, sanitary towels and panty liners as well.

Although only preferred embodiments are specifically illustrated and described herein, it will be appreciated that many modifications and variations of the present invention are possible in light of the above teachings and within the purview of the appended claims without departing from the spirit and intended scope of the invention.

1. An absorbent article with a longitudinal direction and a transverse direction comprising an absorption body which is enclosed in a cover and comprises at least one absorption layer, the cover comprising a liquid-permeable surface and a liquid-impermeable surface,
wherein the absorption body comprises a first absorbent part and a second absorption part which is separate and displaced in a longitudinal direction from the first absorption part,
wherein the first absorption part has a tongue-shaped portion adapted to be arranged in between two legs extending essentially in the longitudinal direction on the second absorption part,
wherein fold indications are formed in the absorption body between the tongue-shaped portion of the first absorption part and at least each leg of the second absorption part.

2. The absorbent article according to claim 1, wherein the article comprises two end portions and a crotch portion, wherein the crotch portion is located between the end portions and has a smaller size in a transverse direction than the end portions, and wherein the fold indications in the absorption body are arranged at least mainly in the crotch portion.

3. The absorbent article according to claim 1, wherein the two legs extending in the longitudinal direction on the second absorption part have a length of 3-30 cm.

4. The absorbent article according to claim 1, wherein the two legs extending in the longitudinal direction on the second absorption part have a length of 5-25 cm.

5. The absorbent article according to claim 1, wherein the two legs extending in the longitudinal direction on the second absorption part have a length of 7-20 cm.

6. The absorbent article according to claim 1, wherein the tongue-shaped portion of the first absorption part has a length of 3-30 cm.

7. The absorbent article according to claim 1, wherein the tongue-shaped portion of the first absorption part has a length of 5-25 cm.

8. The absorbent article according to claim 1, wherein the tongue-shaped portion of the first absorption part has a length of 7-20 cm.

9. The absorbent article according to claim 1, wherein there is a distance of 0-20 mm between the tongue-shaped portion of the first absorption part and each leg of the second absorption part at least at outer parts of the legs.

10. The absorbent article according to claim 1, wherein there is a distance of 2-15 mm between the tongue-shaped portion of the first absorption part and each leg of the second absorption part at least at outer parts of the legs.

11. The absorbent article according to claim 1, wherein there is a distance of 3-10 mm between the tongue-shaped portion of the first absorption part and each leg of the second absorption part at least at outer parts of the legs.

12. The absorbent article according to claim 9, wherein the outer parts of the legs extend in the longitudinal direction and form the whole of the length of the fold indications.

13. The absorbent article according to claim 9, wherein the outer parts of the legs extend in the longitudinal direction and form about a half of the length of the fold indications, displaced at the outermost tip of the legs.

14. The absorbent article according to claim 9, wherein the outer parts of the legs extend in the longitudinal direction and form about a third of the length of the fold indications, displaced at the outermost tip of the legs.

15. The absorbent article according to claim 1, wherein the first absorption part is locked in a predetermined position in relation to the second absorption part by a stabilizing layer.

16. The absorbent article according to claim 15, wherein the stabilizing layer is connected firmly to the first absorption part and the second absorption part.

17. The absorbent article according to claim 16, wherein the stabilizing layer is selected from the group comprising a distance layer between the first absorption part and the second absorption part and the liquid-permeable surface of the cover, the liquid-permeable surface of the cover, the liquid-impermeable surface of the cover, both cover layers, a strengthening layer, a liquid receiving layer, and an additional absorption layer.

18. The absorbent article according to claim 1, wherein the article is adapted for use as a children's diaper and wherein the fold indications have a length of 3-20 cm.

19. The absorbent article according to claim 18, wherein the fold indications have a length of about 15 cm.

20. The absorbent article according to claim 1, wherein the article is adapted for use as a children's diaper and wherein a distance in the traverse direction, measured at the termination points, between the fold indications is 1-5 cm.

21. The absorbent article according to claim 20, wherein the distance in the traverse direction, measured at the termination points, between the fold indications is about 3 cm.

22. The absorbent article according to claim 1, wherein the article is adapted for use as an adult's diaper and wherein the fold indications have a length of 3-30 cm.

23. The absorbent article according to claim 22, wherein the fold indications have a length of about 20 cm.

24. The absorbent article according to claim 1, wherein the article is adapted for use as an adult's diaper and wherein a distance in the traverse direction, measured at the termination points, between the fold indications is 1-7 cm.

25. The absorbent article according to claim 24, wherein the distance in the traverse direction, measured at the termination points, between the fold indications is about 4 cm.

26. The absorbent article according to claim 1, wherein the article is adapted for use as a sanitary towel or incontinence towel and wherein the fold indications have a length of 5-20 cm.

27. The absorbent article according to claim 26, wherein the fold indications have a length of about 10 cm.

28. The absorbent article according to claim 1, wherein the article is adapted for use as a sanitary towel or incontinence towel and wherein a distance in the traverse direction, measured at the termination points, between the fold indications is 1-5 cm.

29. The absorbent article according to claim 28, wherein the distance in the traverse direction, measured at the termination points, between the fold indications is about 2.5 cm.

30. The absorbent article according to claim 1, wherein the first absorption part is located in a rear end portion of the article and is adapted to face backwards on a wearer during use, and the second absorption part is located in a front end portion of the article and is adapted to face forwards on the wearer during use.

31. The absorbent article according to claim 30, wherein the first absorption part and the second absorption part comprise the same absorbent material.

32. The absorbent article according to claim 30, wherein the second absorption part comprises absorbent material adapted for taking up at least one of urine, other viscous liquids, and menstrual fluids and discharges.

33. The absorbent article according to claim 30, wherein the first absorption part comprises absorbent material adapted for taking up at least one of feces, other viscous liquids, and menstrual fluids and discharges.

34. The absorbent article according to claim 1, wherein the first absorption part is located in a front end portion of the article and is adapted to face forwards on a wearer during use, and the second absorption part is located in a rear end portion of the article and is adapted to face backwards on the wearer during use.

35. The absorbent article according to claim 34, wherein the first absorption part and the second absorption part comprise the same absorbent material.

36. The absorbent article according to claim 34, wherein the first absorption part comprises absorbent material adapted for taking up at least one of urine, other viscous liquids, and menstrual fluids and discharges.

37. The absorbent article according to claim 34, wherein the second absorption part comprises absorbent material adapted for taking up at least one of feces, other viscous liquids, and menstrual fluids and discharges.

38. The absorbent article according to claim 1, wherein an inner edge of the legs of the second absorption part comes in towards a central line so as thus to form a U-shaped edge
wherein the U-shaped edge runs essentially edge to edge with an outer edge of the tongue-shaped portion of the first absorption part.

39. The absorbent article according to claim 1, wherein an inner edge of the legs of the second absorption part comes in towards a central line so as thus to form a U-shaped edge
wherein there is a distance between the U-shaped edge of the second absorption part and an outer edge of the tongue-shaped portion of the first absorption part of 1-20 mm along the whole tongue-shaped portion.

40. The absorbent article according to claim 1, wherein an inner edge of the legs of the second absorption part comes in towards a central line so as thus to form a U-shaped edge
wherein there is a distance between the U-shaped edge of the second absorption part and an outer edge of the tongue-shaped portion of the first absorption part of 2-15 mm along the whole tongue-shaped portion.

41. The absorbent article according to claim 1, wherein an inner edge of the legs of the second absorption part comes in towards a central line so as thus to form a U-shaped edge
wherein there is a distance between the U-shaped edge of the second absorption part and an outer edge of the tongue-shaped portion of the first absorption part of 3-10 mm along the whole tongue-shaped portion.

42. The absorbent article according to claim 39, wherein the first absorption part is located in a front end portion of the article and is adapted to face forwards on the wearer during use, and the second absorption part is located in a rear end portion of the article and is adapted to face backwards on the wearer during use.

43. The absorbent article according to claim 1, wherein the tongue-shaped portion of the first absorption part overlaps at least a part of the second absorption part.

44. The absorbent article according to claim 43, wherein the tongue-shaped portion of the first absorption part overlaps a part of the second absorption part so that a double layer is formed in at least an area corresponding to a wetting zone of the absorbent article.

45. A method for producing an absorption body for use in an absorbent article, the method comprising:
shaping a first absorption part of the absorbent body to have a tongue-shaped portion;
shaping a second absorption part of the absorbent body to have two legs extending essentially in a longitudinal direction;
positioning the first absorption part separately from the second absorption part such that the tongue-shaped portion of the first absorption part is arranged completely or partly in between the two legs extending essentially in the longitudinal direction of the second absorption part; and
forming fold indications in the absorption body between the tongue-shaped portion of the first absorption part and each leg of the second absorption part.

46. The method according to claim 45, wherein at least a part of an inner edge of the legs of the second absorption part is positioned edge to edge with an outer edge of the tongue-shaped portion.

47. The method according to claim 45, wherein the first absorption part is arranged such that the tongue-shaped portion of the first absorption part overlaps at least a part of the second absorption part.

48. The method according to claim 45, wherein an inner edge of the legs of the second absorption part comes in towards a central line so as thus to form a U-shaped edge, wherein a first absorption part is arranged such that there is a distance between a U-shaped edge of the second absorption part and an outer edge of the tongue-shaped portion of the first absorption part of 1-20 mm.

49. The method according to claim 48, wherein there is a distance between the U-shaped edge of the second absorption part and an outer edge of the tongue-shaped portion of the first absorption part of 2-15 mm.

50. The method according to claim 48, wherein there is a distance between the U-shaped edge of the second absorption part and an outer edge of the tongue-shaped portion of the first absorption part of 3-10 mm.

51. The method according to claim 45, comprising the additional step of fixing the shape of the absorption body after positioning of the first absorption part and the second absorption part in relation to one another by joining the absorption body together with a stabilizing layer.

52. The method according to claim 45, comprising the additional step of fixing the shape of the absorption body after positioning of the first absorption part and the second absorption part in relation to one another by activating thermoplastic fibers included in the absorption body to a layer included in the absorbent article, by heating and subsequently cooling the absorption body.

53. An absorbent article with a longitudinal direction and a transverse direction comprising an absorption body which is enclosed in a cover and comprises at least one absorption layer, the cover comprising a liquid-permeable surface and a liquid-impermeable surface,
wherein the absorption body comprises a first absorbent part and a second absorption part which is separate and displaced in a longitudinal direction from the first absorption part,
wherein the absorbent body further comprises means for forming fold indications between the first absorbent part and the second absorbent part.

54. The absorbent article of claim 53, further comprising means for forming a curvature of the absorbent body in a crotch portion of the absorbent article.

55. The absorbent article of claim 53, further comprising means for forming a channel in the absorbent body.

56. The absorbent article of claim 53, further comprising means for providing increased absorption capacity in a wetting zone of the absorbent article.

57. The absorbent article of claim 53, wherein the first absorption part is locked in a predetermined position in relation to the second absorption part by a stabilizing means.
